(19) United States
(12) Patent Application Publication
(10) Pub. No.: US 2026/0225999 A1
(43) Pub. Date: Aug. 6, 2026

(54) VISIBLE LIGHT DRIVEN CO2 VALORIZATION

(71) Applicant: THE BOARD OF REGENTS FOR THE OKLAHOMA AGRICULTURAL AND MECHANICAL COLLEGES, Stillwater, OK (US)

(72) Inventors: Jimmie D WEAVER, Stillwater, OK (US); Tim SCHOCH, Stillwater, OK (US)

(73) Assignee: THE BOARD OF REGENTS FOR THE OKLAHOMA AGRICULTURAL AND MECHANICAL COLLEGES, Stillwater, OK (US)

(21) Appl. No.: 19/150,383

(22) PCT Filed: Feb. 29, 2024
(86) PCT No.: PCT/US2024/017844

§ 371 Date: Jul. 23, 2025

Related U.S. Application Data
(60) Provisional application No. 63/449,488, filed on Mar. 2, 2023.

(51) Int. Cl. C07C 269/04; C07C 269/06; C07C 271/10

(52) U.S. Cl. CPC C07C 269/04; C07C 269/06; C07C 271/10

(57) ABSTRACT

A method of carbamate ester synthesis wherein a first component is reacted with a cyclohexene by the addition of CO₂, a photocatalyst, and light to produce an arylcyclohexyl carbamate ester. The first component is an amine, a carbamate salt produced from the amine, or a mixture thereof. In some embodiments, the method further includes treating the arylcyclohexyl carbamate ester with a base in the presence of an alcohol to produce an alkane carbamate ester and an arylcyclohexanol. Further, the method can include dehydrating the arylcyclohexanol in the presence of an acid to regenerate the arylcyclohexene.

### CROSS REFERENCE TO RELATED APPLICATION

The present application claims priority to U.S. Provisional Application No. 63/449,488 filed on Mar. 2, 2023, which is incorporated herein.

### STATEMENT REGARDING FEDERALLY SPONSORED RESEARCH OR DEVELOPMENT

This invention was made with government support under Contract Number R35GM139613 awarded by the National Institutes of Health. The government has certain rights in the invention.

### BACKGROUND

Carbamate esters, or urethanes, are a key functional group in a myriad of medicinal and agricultural compounds where they function broadly as hydrolytically and proteolytically stable peptide mimetics. They can be found in antibiotics such as Linezolid, antiretrovirals such as Ritonavir, cancer treatments like Irinotecan, and a range of pesticides bearing the methylamine carbamate motif. The urethane functionality is also in high demand globally for polymeric materials, such as methylene diphenyl diisocyanate and isophorone diisocyanate-derived polyurethanes. Materials like these have markets valued at well over 20 billion USD in recent years, creating a need for energy-efficient means of production. Urethanes are predominantly produced in two ways which depend on the origin of the carbonyl C1 unit: carbon monoxide or carbon dioxide.

A typical carbon monoxide process is illustrated in FIG. 1. Carbon monoxide, owing to its toxic nature and need of external oxidant is most often converted to other energy-rich intermediates like phosgene (also highly toxic) en route to urethanes. Although these intermediates can produce a wide variety of urethanes in high yields, they exact a high energy toll in their own production. It bears mentioning that CO itself is produced primarily from fossil fuels through energetically intense processes like steam-methane reforming reaction which takes place at 700-1000° C.

Carbon dioxide, on the other hand, is an especially attractive C1 source as it is, already in the proper oxidation state, relatively innocuous, cheap, and abundant. CO₂ presents a critical challenge to incorporate into value-added products with its inherent thermodynamic stability. As illustrated in FIG. 2, strategies to date still tend toward reliance on reactive electrophiles, such as alkyl halides, epoxides, dialkyl carbonates, tosylhydrazones, and on dehydrating agents via Mitsunobu and Swern-like conditions as the driving force for their reactions.

A strategy, reported in 1977 by Yoshida and Inoue, demonstrated an approach to generate the energetic electrophile in situ using ethyl vinyl ether as a base (see FIG. 3). The hope was that the vinyl ether would be protonated by carbamic acid formed in situ to simultaneously generate an activated oxo-carbenium ion and activated carbamate anion. Unfortunately, the reaction relied on CO₂ pressures and temperatures that required an autoclave over the course of multiple days to drive the reaction forward, and still had no yields exceeding 11%. This is likely a result of the modest basicity of the vinyl ether and an unfavorable proton transfer with ammonium carbamate salts.

Accordingly, there is a need for improved process to produce carbamate esters with high yields and without the necessity of harsh chemicals or high temperatures and or pressures.

### SUMMARY

Disclosed herein is a means of generating the reactive electrophilic species in situ under mild conditions. In the process of this disclosure, styrenic alkenes (such as arylcyclohexene) are treated to blue light irradiation, they are excited to short-lived biradicals and are granted brief rotational freedom and rapidly relax down to the ground state cis and trans-isomers. The strain energy confers greatly enhanced stereoselective dienophilicity, tendency toward [2+2] cycloadditions, and basicity. These trans-cyclohexenes, generated with visible light, are sufficiently basic to undergo protonation by carbamic acid or its ammonium salt and subsequent rapid trapping of the incipient carbenium ion, to ultimately yield the urethane product (carbamate ester product). The use of visible light over the precedented ultraviolet mitigates side-product formation and improve functional group tolerance. This approach provides the first example of CO₂ valorization using visible light as an indirect driving force through the energy currency of molecular strain.

### BRIEF DESCRIPTION OF THE DRAWINGS

The drawings included in this disclosure illustrate certain aspects of the embodiments described herein. However, the drawings should not be viewed as exclusive embodiments. The subject matter disclosed herein is capable of considerable modifications, alterations, combinations, and equivalents in form and function, as well be evident to those skilled in the art with the benefit of this disclosure.

FIG. 1 is an illustration of a prior art process using CO to produce a carbamate ester product.

FIG. 2 is an illustration of a prior art process using CO₂ to produce a carbamate ester product.

FIG. 3 is an illustration of another prior art process using CO₂ to produce a carbamate ester product.

FIG. 4 is a simplified illustration of the process of this disclosure.

FIG. 5 is a simplified illustration a further process according to this disclosure with recovery of the arlycycohexene.

FIG. 6 is a diagram illustrating various amines tested in a process according to this disclosure.

FIG. 7 is a graph illustrating reaction rate of different cyclohexenes used in a process of this disclosure.

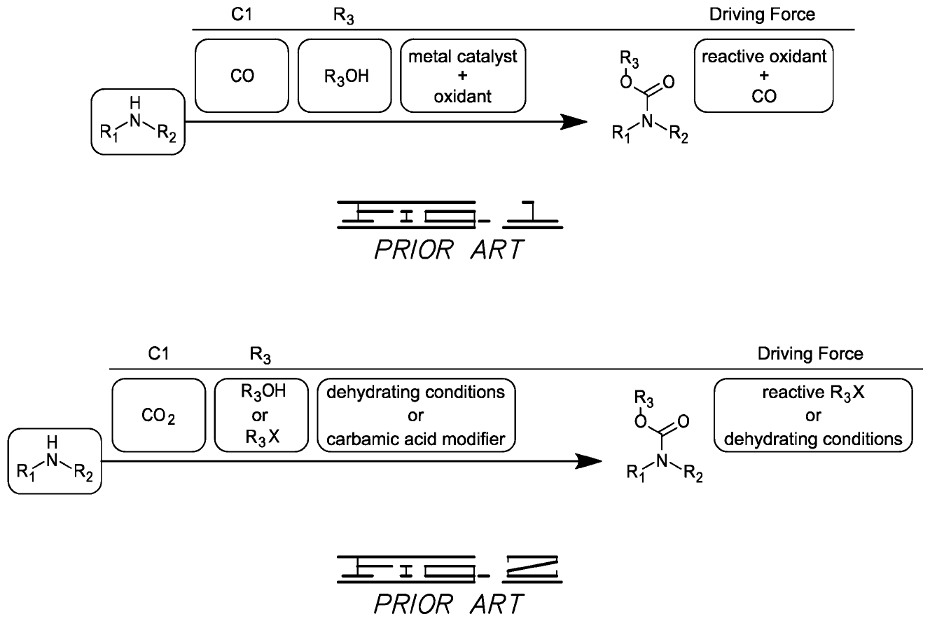

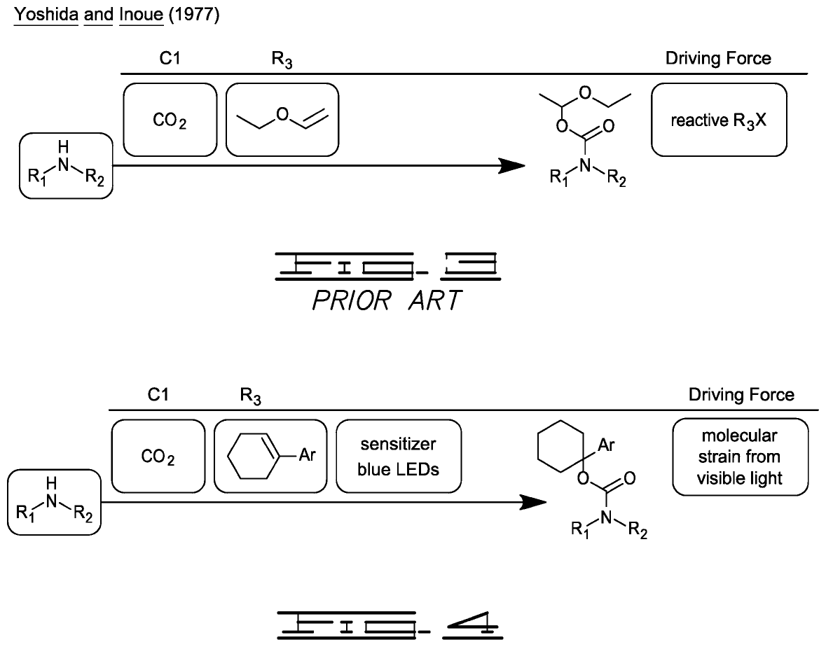

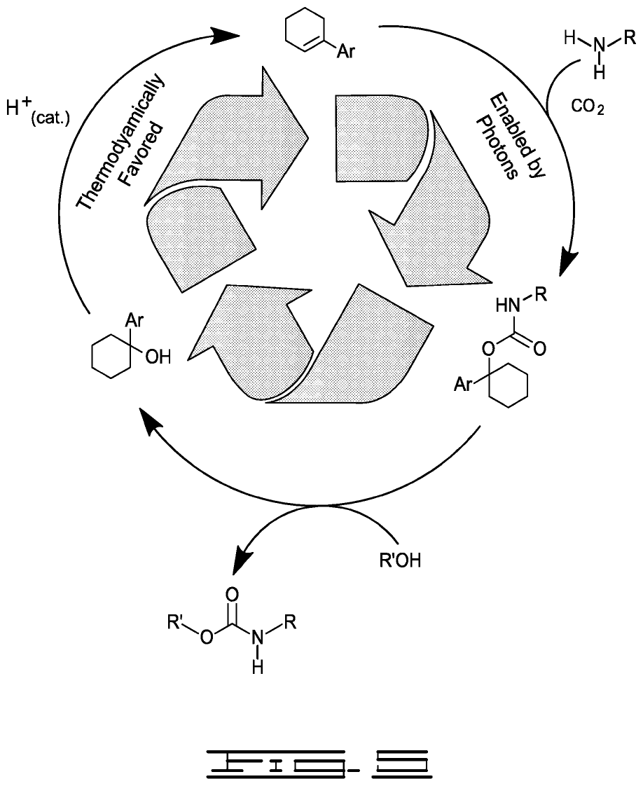

### DESCRIPTION

The present disclosure may be understood more readily by reference to this description as well as to the examples included herein. In addition, numerous specific details are set forth in order to provide a thorough understanding of the embodiments and examples described herein. However, those of ordinary skill in the art will understand the embodiments and examples described herein can be practiced without these specific details. In other instances, methods, procedures and components have not been described in detail so as not to obscure the related relevant feature being described. Also, the description is not to be considered as limiting the scope of the embodiments described herein.

As used herein, the following terms have the following meanings.

“Carbamic acid” refers to an acid compound of the form:

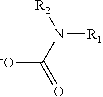

Where R₁ and R₂ are aliphatic functional groups or hydrogen. Generally, in this disclosure either R₁ or R₂ may be a hydrogen but not both; thus at least one of R₁ or R₂ will be an organic functional group; though R₁ and R₂ may be part of the same aliphatic ring.

Further, in this disclosure carbamic acid can be part of a preformed carbamic salt or the carbamic acid may be formed from an aliphatic amine. The preformed salt will be the carbamic acid with its corresponding ammonium base, and when formed from an aliphatic amine in situ in the process of this disclosure, carbamic acid will be formed along with its corresponding ammonium base of the form:

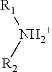

Where R₁ and R₂ are the same as in the corresponding carbamic acid.

Accordingly, when referring to carbamate salt, this disclosure means the carbamic acid along with its corresponding ammonium base, whether the carbamate salt is preformed or formed in situ during the process of this disclosure. Thus, the carbamate salt has the formula:

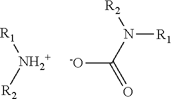

“Carbamate ester” or “urethane” refers to a compound of the structural formula:

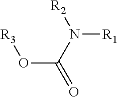

Where R₁ and R₂ are an organic function group or hydrogen, and R₃ is an organic functional group. In the carbamate esters formed herein, either R₁ or R₂ may be a hydrogen but not both; thus at least one of R₁ or R₂ will be an organic functional group. Additionally, R₁ and R₂ may form part of the same organic ring, which also contains the N atom. Typically, R₁ and R₂ will be aliphatic functional groups or hydrogen, and can form part of the same aliphatic ring. As will be apparent from the discussion herein, in the arylcyclohexyl carbamate esters formed under this disclosure, R₃ will be an arylcyclohexyl group based on the arylcyclohexene used, in the alkane carbamate esters formed under this disclosure, R₃ will be an alkane group based on the alcohol used.

“Photocatalyst”, “sensitizer” or “photosensitizer” as used herein refers to light absorbers that alter the course of a photochemical reaction. A sensitizer or photosensitizer is a molecule or cluster/colloidal particle or a solid, which absorbs electromagnetic radiation at a certain wavelength and transfers that energy in electronic form (via electron transfer, proton transfer, resonance energy transfer (Dexter or Forster mechanism), hydrogen abstraction, etc.) to a neighboring molecule, which otherwise cannot absorb the electromagnetic radiation directly by itself. At the end of this process, the photosensitizer returns to its ground state, where it remains chemically intact, poised to absorb more light. Generally, photosensitizers absorb electromagnetic radiation consisting of infrared radiation, visible light radiation, and ultraviolet radiation and transfer absorbed energy into neighboring molecules. The photosensitizers useful in the processes of this disclosure are typically ones that absorb blue or violet light, as further discussed below.

Typically, as used herein the photocatalyst or sensitizers used are ones which catalyze the reaction for the herein described synthesis of carbamate ester by absorbing visible light (380-700 nm), and specifically blue or violet light (about 380-500 nm). Advantageously, this results in a reaction easier to facilitate than the use of photocatalyst that must be excited with UV-irradiation. Generally, the absorption will be blue or violet light of 495 nm wavelength or less, or 495-380 nm. For example, photocatalyst that absorb light of 455 nm wavelength or less, or from 455 nm to 380 nm, are particularly effective.

Blue light has a wavelength of 450 nm to 500 nm, and more narrowly from 450 nm to 495 nm. Violet light has a wavelength of 380 nm to less than 450 nm. As used herein, “blue light” will typically mean blue or violet light as described above, and “blue light (450+)” will refer to only blue light. That said, those skilled in the art will understand that there is no standard cut off line between blue and violet light; for example, some sources report 400 nm as being blue light and others as being violet light. Applicant is expressing disclosing all the lesser ranges included between 380 nm and 500 nm, and if narrowing blue light to one of those will use a convention of “blue (X-Y)” where X is the shorter wavelength and Y is the longer wavelength of the blue light range. For example, blue (435-495), would mean light with a wavelength from 435 nm to 495 nm.

In other aspects, the photocatalyst used are ones that have an emissive energy of at least 56 kcal/mol, more typically of at least 56.4 kcal/mol, and even more typically of at least 58 kcal/mol, or even at least 60 kcal/mol. For example, the photocatalyst used herein can be (dFppy)₃, 2CzPN (4,5-di(9H-carbazol-9-yl) phthalonitrile), p-2CzPN, Ir(Ftbppy)₃, and Ir(dFCF₃ppy)₂(dtbbpy)PF₆.

This disclosure is directed at a method for urethane synthesis by addition of carbon dioxide (CO₂) and amines to photoactivated olefins using a styrenic alkenes (such as styrenic alkenes) under mild conditions, such as 0.1 MPa CO² at 7° C. (280 K). The method avoids the use of phosgene and carbon monoxide (CO), or any reagents directly or indirectly derived from them.

As indicated, the disclosed process produces urethane under mild conditions; however, it can be carried out under a wide range of conditions based on the particular components used. For example, if dichloromethane (DCM) is used as the solvent, the reaction may be carried out in the temperature range of −60° C. to 37° C. at 1 atm pressure. The synthesis reaction being limited by the boiling point of DCM at the higher end. Thus, if higher pressures are used, then the higher temperatures can be used. Thus, for example the synthesis may be carried out from of −60° C. to 100° C. at from 0.04 atm to 4 amt CO₂. Advantageously, the current synthesis can be carried out at near standard temperature and pressure; that is, from −10° C. to 40° C. and from 0.8 atm to 1.2 atm. Accordingly, when “near standard temperature and pressure” or “near STP” is used herein, it refers to these ranges.

CO₂ aminoalkylation using olefins and amines represents a means of obtaining carbamate esters, or urethanes, except that this reaction is highly endergonic at standard temperature and pressure (STP) or at near STP. Consequently, the conventional methods of flexible urethane preparation from amines involve, at some point in the synthesis, high energy molecules to make the process exergonic, which are themselves typically also harsh, cumbersome, and toxic.

Herein disclosed is a compellingly mild and convenient method of CO₂ aminoalkylation which uses visible light energy to drive this endergonic process (+25 kcal/mol at STP). The disclosed method uses styrenic alkenes (such as arylcyclohexene) which converts much of the light photon's energy to strain energy upon olefin isomerization. This strain energy results in dramatically increased basicity of the arylcycloalkene allowing for protonation by the in situ formed carbamic acid which occurs spontaneously at STP. The method boasts water tolerance and use of CO₂ near standard temperature and pressure. Additionally, the method includes the conversion of the resulting carbamates to other carbamates, amides, and ketones by the extrusion of arylcyclohexanol. Also, the arylcyclohexanol can be catalytically converted back to the parent alkene by the use of an acid catalyst.

Turning now to FIG. 4, a simplified diagram of a reaction of the process can be seen wherein an amine is converted to a carbamate ester having the same functional groups (R₁ and R₂) as the amine. Broadly, the amine is converted by exposure to CO₂ and blue light in the presence of an arylcyclohexene and a photocatalyst.

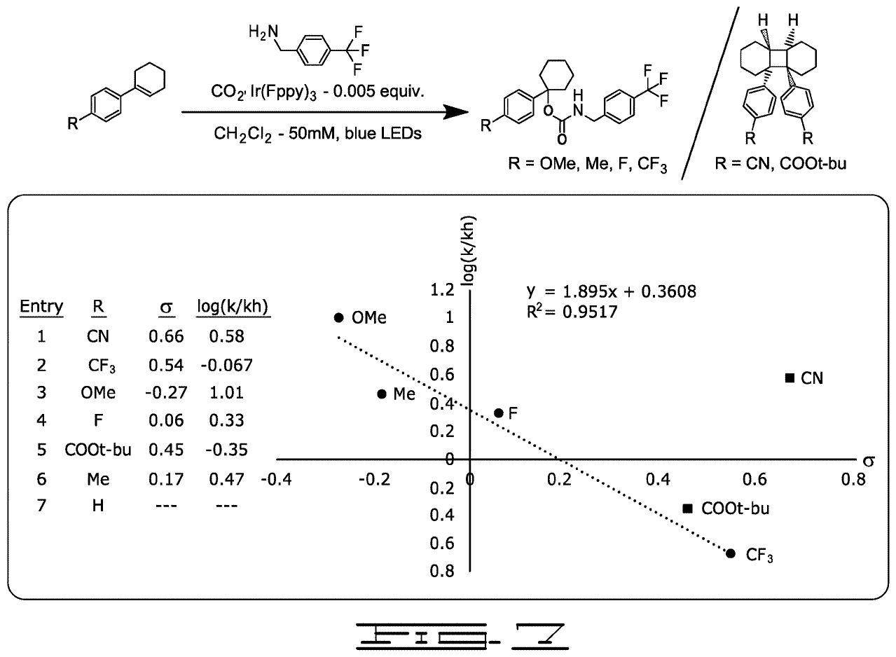

The amine is typically a primary or secondary aliphatic amine:

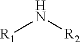

Where R₁ and R₂ are aliphatic functional groups or hydrogen. Generally, in this disclosure either R₁ or R₂ may be a hydrogen but not both; thus at least one of R₁ or R₂ will be an organic functional group; though R₁ and R₂ may be part of the same aliphatic ring (see for example amine #14 or #18 in FIG. 6).

In the process, the amine will be converted to a carbamate salt, as defined above. Accordingly, some embodiments can use the carbamate salt in place of the amine.

The arycyclohexane will generally be of the form:

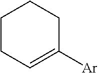

Where Ar is an aryl functional group derived from an aromatic ring, such as phenyl or naphthenyl.

The carbamate ester or urethane produced will be arylcyclohexyl carbamate ester having the form:

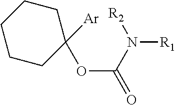

Where the R₁ and R₂ functional groups come from the amine (or carbamate salt). As a result of the above described process, the R₃ functional group is a cyclohexane function group containing the Ar group, which comes from the arylcyclohexene.

Generally, the process will be carried out in the presence of an organic solvent. For example, the organic solvent can be a halogenated solvent. For example, the organic solvent can be an acetonitrile, dimethyl sulfoxide (DMSO) or a halogenated solvent, such as dichloromethane or dichloroethane. For example, the organic solvent can be dichloroethane, dichloromethane or acetonitrile.

In the process, the amine and arylcyclohexene will typically be present in a ratio of from 1 to 2, or 2 to 1, and more typically from 1 to 1.05 of mole of amine per mole of arylcyclohexene. For example, the ratio would typically be about 1.05. For example, the amine could be present in an amount from 100 mol % to 95 mol % and the arylcyclohexene could be present in an amount from 100 mol % to 105 mol %. The process can be practiced outside these ranges, but doing so can increase side reactions.

While the process can be carried out in a pressurized environment, it more advantageously can be carried out at ambient pressure or atmospheric pressure. The process can be carried out in a CO₂ environment; however, it can also be carried out in air in which CO₂ is present in an amount from about 2% up to 100%, or from 4% to 99%, of the air. Additionally, the process can be carried out in a combination of inert gases and CO₂, with the CO₂ present in an amount from about 2% up to 100%, or from 4% to 99% of the combination. However, it is currently believed that O₂ can adversely affect the reaction, thus either a CO₂ or CO₂/inert gas combination is presently preferred. For example, the process can be carried in 0.1 MPa CO₂ (about 1 atm).

The photocatalyst can be present in an amount from 0.02 mol % to about 5 mol %, but typically from 0.05 mol % to 2 mol %, and more typically from 0.1 mol % to 1 mol %.

The organic solvent will typically be present in an amount from 25 mM to 500 mM, and more typically from 30 mM to 400 mM, or from 40 mM to 200 mM.

The method can further include steps to convert the arylcyclohexyl carbamate ester to other carbonate esters. For example, transcarbamoylations can be used to convert the arylcyclohexyl carbamate ester to alkane carbamate ester. For example, the arylcyclohexyl carbamate ester can be mixed with a base in the presence of an alcohol to produce an alkane carbamate ester and an arylcyclohexanol, wherein the alkane group comes from the alcohol and the aryl group comes from the arylcyclohexyl carbamate ester. For example, the mixing can occur at near STP; for example, the mixing can occur at room temperature (ambient temperature).

Typically, the alcohol will have a hydroxyl group and an alkane group. For example, the alcohol can be a primary or secondary alcohol. For example, the alcohol can be cinnamyl alcohol, β-citronellol, or 2-propanol. The base can be any suitable base to promote the transcarbamoylation reaction. For example, the conjugate acid of the base can have a pH from 12 to 50. For example, the base can be NaOH, KOt-bu (potassium tert-butoxide-[(CH3) 3COK]ₙ), or KOH, which are efficient catalyst to promote the transcarbamoylation reaction between urethanes and a variety of primary and secondary alcohols under mild conditions.

The arylcyclohexanol formed as the byproduct can undergo dehydration to regenerate the cycloalkene. For example, the arylcyclohexanol can undergo gentle heating (for example, approximately 60° C. to 70° C.) with an acid, such as p-toluenesulfonic acid).

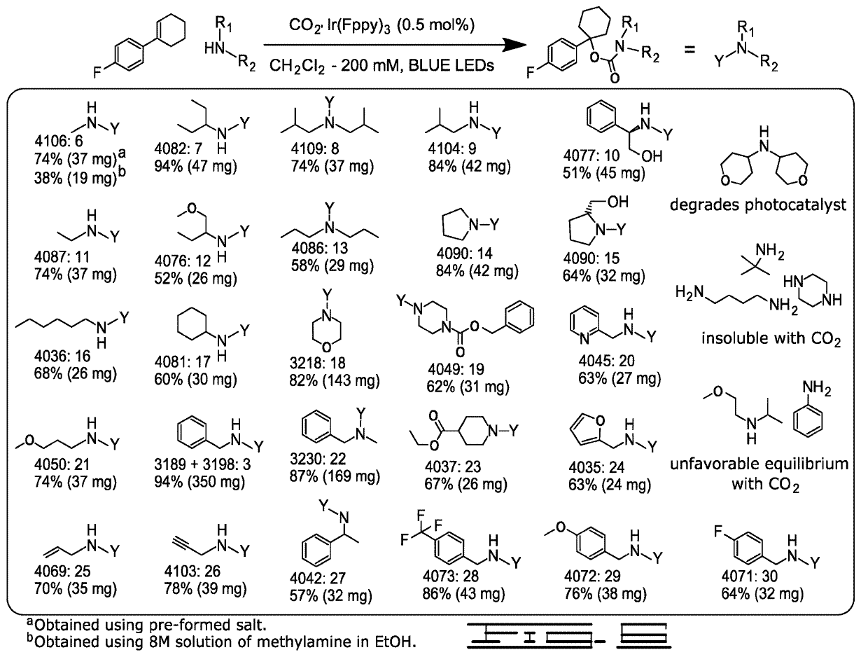

An example of a process for producing an alkane carbamate ester from an amine, and for recycling the arylcyclohexanol used in the process is graphically illustrated in FIG. 5.

Additionally, the urethane products can be converted to the corresponding amides and ketones using Grignard or organolithium reagents, respectively. In such conversions, an arylcyclohexanol is formed as the byproduct. Conveniently, the dehydration of this alcohol is thermodynamically favored, and can be used to regenerate the cycloalkene, as discussed above.

### EXAMPLES

### Example 1

A control was run with 200 mM 4-fluorophenylcyclohexene (as shown below).

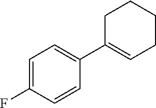 [SMILES: Fc1ccc(C2=CCCCC2)cc1]

The cyclohexane was irradiated with blue light (using blue LEDs) in the presence of catalytic amounts (0.2 mol %) of Ir(Fppy)₃ in 0.1 MPa CO₂ and 200 mM of dichloromethane (CH₂Cl₂) solvent. The reaction was carried out at 7° C. (280 K)—the reactions of Examples 2-37 were carried out between 5° C. and room temperature. The fluorinated photocatalyst and 4-fluorophenylcyclohexene enabled convenient reaction monitoring by Flurine-19 nuclear resonance spectroscopy (¹⁹F NMR). The reaction was monitored for conversion for urethane synthesis and for conversion to the predominant side reaction of 2+2 photodimerization of the cyclohexane obtaining the cyclobutane product (2+2 cycloadduct), structural formula:

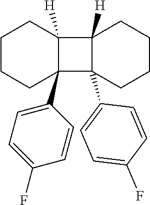

Additionally, the reaction was monitored for the amount of cyclohexane remaining. The reaction was run over the course of 2 days. The results (Table 1) show that no urethane was produced instead there was 91% conversion to the cyclobutene product with 2% cyclohexene remaining.

This is somewhat anticipated reactivity based on prior work with cycloheptene homologs, wherein the trans-cycloheptene forms a cyclobutane dimer.

### Example 2

A reaction was carried out using benzylammonium benzylcarbamate salt:

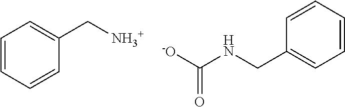

The 50 mM benzylammonium benzylcarbamate was combined with 50 mM 4-fluorophenylcyclohexene. The mixture was irradiated with blue light in the presence of catalytic amounts (0.2 mol %) of Ir(Fppy)₃ in argon-saturated dichloromethane with no CO₂ present. The reaction was carried out at 7° C. (280 K). The fluorinated photocatalyst and 4-fluorophenylcyclohexene enabled convenient reaction monitoring by Flurine-19 nuclear resonance spectroscopy (19F NMR). The reaction was monitored by 19F NMR for conversion for urethane synthesis to:

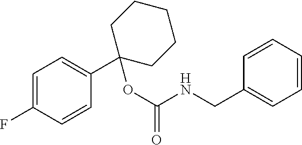 [SMILES: O=C(NCc1ccccc1)OC1(c2ccc(F)cc2)CCCCC1]

Additionally, the reaction was monitored for the cyclobutane product (2+2 cycloadduct), and monitored for any remaining cyclohexene. The reaction was monitored until it plateaued (the reactions of Examples 2 to 37, all were monitored for at least 3 hours; typically, from 3 hours to 7 hours; however, as indicated below some went longer).

A slow conversion of cyclohexene to the corresponding urethane was observed but the conversion plateaued at approximately 49% urethane within 12 hours, as shown in Table 1. An assessment of the mass balance showed that besides the urethane product, there was mostly unreacted starting material with little of the 2+2 cycloadduct product. Notably, no amine alkylation products were detected.

### Example 3

A second reaction was carried out using benzylammonium benzylcarbamate in its salt form. This reaction was carried out as per Example 2, except that the argon atmosphere was replaced with CO₂.

As shown in Table 1, the use of CO₂ accelerated the reaction, consuming 98% of the cyclohexene within 4 hours, with 87% of the product identified as urethane.

### Example 4

In another set of experiments under CO₂ atmosphere, 50 mM benxylamine was instead of benzylammonium benzylcarbamate in its preformed salt form. The conditions were otherwise in accordance with Example 3.

The results showed that using preformed salt was indistinguishable from its in situ generation starting from benzylamine, both in terms of reactivity under blue light (result shown in Table 1) and by comparison of ¹³C NMR spectra of reaction mixtures collected prior to exposure to the blue LEDs. As such, subsequent experiments reported herein were performed with benzylamine rather than the preformed salt.

### Example 5

This Example 5 was carried out as per Example 4, except in the argon was used instead of CO₂. As shown in Table 1, in the absence of CO₂ there was no urethane production and less than <2% conversion to cyclobutene. There was also a slow formation of orange color which was correlated to photocatalyst degradation, as observed in the 19F NMR spectrum.

### Example 6

This Example 6 was carried out as per Example 3, except no photocatalyst was present and no light was used. As reported in Table 1, this resulted in no conversion with all the cyclohexene remaining at the end of the test.

TABLE 1

| | | | | % |
|---|---|---|---|---|
| | | | | Cyclobutene |
| | | % | % | (2 + 2 |
| | | Cyclohexene | Urethane | cycloadduct) |
| Example 1 | No benzylamine or | 2 | 0 | 91 |
| | preformed salt; | | | |
| | CO₂ | | | |
| Example 2 | Preformed salt; | 44 | 49 | 3 |
| | Argon | | | |
| Example 3 | Preformed salt; | 2 | 87 | 5 |
| | CO₂ | | | |
| Example 4 | Benzylamine; | 0 | 94 | 3 |
| | CO₂ | | | |
| Example 5 | Benzylamine; | 98 | 0 | 2 |
| | Argon | | | |
| Example 6 | No light; | 100 | 0 | 0 |
| | no photocatalyst | | | |

As illustrated by Examples 1-6, the reaction to produce cyclobutene from the cyclohexene is readily mitigated through both dilute concentration and, more significantly, the proton transfer between species the benzylamine/preformed salt and the cyclohexene en route to urethane. Thus, the successful urethane synthesis by addition of CO₂ and amines to photoactivated olefins under blue light has been shown.

### Examples 7-13

Solvent variation was carried out, with the knowledge that at least the alkene geometrical isomerization step is viable in many solvents. Tests were carried out in accordance with Example 4 but using different solvents from the dichloromethane solvent used in Example 4. The solvent used and the results are shown in Table 2.

TABLE 2

| | | | | % Cyclobutene |
|---|---|---|---|---|
| | | % | % | (2 + 2 |
| | Solvent | Cyclohexene | Urethane | cycloadduct) |
| Example 7 | Dimethylformamide | 4 | 17 | 79 |
| Example 8 | Dimethyl sulfoxide | 0 | 16 | 48 |
| Example 9 | Methanol | 13 | 6 | 0 |
| Example 10 | Tetrahydrofuran | 43 | 44 | 13 |
| Example 11 | Ethyl acetate | 38 | 44 | 17 |
| Example 12 | Toluene | 51 | 19 | 30 |
| Example 13 | Acetonitrile | 5 | 84 | 11 |

Dimethylformamide afforded poor conversion to urethane, being much more conducive to forming the 2+2 cycloadduct. Dimethyl sulfoxide caused the formation of many side products, predominantly the 2+2 cycloadduct, while toluene and ethyl acetate exhibited very sluggish reactivity as the ammonium carbamate salt was only sparingly soluble. Tetrahydrofuran seemed to accelerate photocatalyst degradation in the presence of amine, producing an orange color and halting progress at partial conversion albeit with good selectivity. The best performing solvents were dichloromethane and acetonitrile, both of which ultimately led to excess of 80% conversion to product. Subsequent Examples 14-34 were carried out in dichloromethane.

### Examples 14-16

Examples illustrating the effect of adventitious water, which could lead to undesired tertiary benzylic cyclohexanol, structural formula:

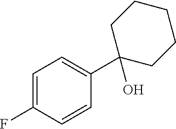 [SMILES: OC1(c2ccc(F)cc2)CCCCC1]

To probe this, the reaction of Example 4 was performed with a range of added water (0 to 50 equivalents) and at 15° C. Table 3 shows the results.

TABLE 3

| | Equivalents | | | % Cyclobutene | % tertiary |
|---|---|---|---|---|---|
| | Added | % | % | (2 + 2 | benzylic |
| | Water | Cyclohexene | Urethane | cycloadduct) | cyclohexanol |
| Example 14 | 1 | 0 | 91 | 3 | 6 |
| Example 15 | 10 | 0 | 88 | 2 | 10 |
| Example 16 | 50 | 0 | 88 | 2 | 10 |

No significant difference in yield was found across this range. The selectivity is likely due to the enhanced nucleophilicity of the negatively charged carbamate anion when compared to the neutral water molecule. Furthermore, the presence of water led to a qualitative attenuation of the photocatalyst turning orange, a process associated with its decomposition, and the end of urethane production. At this temperature (ca. 15° C.), using more than 10 equivalents of water begins to impact reaction homogeneity in dichloromethane, inducing phase separation. The solubility of the CO₂ salts is highly dependent on the amine structure. For instance, in a later reaction using cyclohexylamine instead of benzylamine, a single equivalent of added water led to precipitation of a white solid and a concomitant decrease of desired reactivity. Water seems to have a slight qualitative improvement to reactivity in some cases by enhancing the longevity of the sensitizer.

### Example 17-19

The photocatalyst loading was tested by varying the amount of photocatalyst used. Tests were run in accordance with Example 4, except instead of 0.2 mol % of the Ir(Fppy)₃ photocatalyst, 0.02 mol %, 0.1 mol % and 1 mol % were used. The results are shown in Table 4.

TABLE 4

| | | | | % Cyclobutene |
|---|---|---|---|---|
| | Mol % | % | % | (2 + 2 |
| | Photocatalyst | Cyclohexene | Urethane | cycloadduct) |
| Example 17 | 0.02 | 56 | 33 | 11 |
| Example 18 | 0.1 | 17 | 80 | 2 |
| Example 19 | 1.0 | 0 | 88 | 8 |

As illustrated, the amount of photocatalyst could be dropped to 0.02 mol % before adversely affecting the reaction rate and ultimate conversion according to the examples ranging from 0.02 mol % to 1 mol %. Since Ir(Fppy)₃ worked satisfactorily, it was used in Example 35 at 0.5 mol % loading to account for possible partial degradation over longer reaction times.

### Examples 20-23

Examples 20-23 studied reaction concentration. In anticipation of scaling up synthesis while minimizing solvent usage, the upper limits of concentration was investigated with the benzylamine-Ir(Fppy)₃-4-fluorophenylcyclohexene system of Example 4. A range from 25 to 500 mM was set up in parallel and compared, again, at the 10-minute and four-hour timepoints. Concentrations much higher than this led to issues with insoluble quantities of the in situ generated benzylammonium benzylcarbamate salt. The results are shown in Table 5.

TABLE 5

| | Amount of | | | % Cyclobutene |
|---|---|---|---|---|
| | Dichloromethane | % | % | (2 + 2 |
| | solvent | Cyclohexene | Urethane | cycloadduct) |
| Example | 25 mM, 1 mol % | 48 | 52 | 0 |
| 20 | photocatalyst | | | |
| Example | 50 mM, 1 mol % | 4 | 94 | 1 |
| 21 | photocatalyst | | | |
| Example | 100 mM, 1 mol % | 3 | 90 | 3 |
| 22 | photocatalyst | | | |
| Example | 500 mM, 1 mol % | 5 | 77 | 13 |
| 23 | photocatalyst | | | |

Within this regime, each except the 500 mM sample showed comparable conversion at the 10-minute timepoint. The 500 mM entry was noticeably slower, though it did manage to reach 95% starting material consumption by four hours, albeit with approximately 13% to undesired cyclobutene dimer. The 25 mM sample suffered from incomplete conversion when the photocatalyst degraded—albeit without dimerization, suggesting the ideal concentration to be between these extremes. Subsequent test in Example 35, which investigated the scope of amines amenable to this transformation, were conducted at 150 mM as an attempt to balance mitigation of cycloaddition with the convenience of obtaining isolable yields using NMR tubes as reaction vessels.

### Examples 24-34

Several photocatalysts were surveyed and appraised based on their conversion to product after 10 minutes of irradiation, 4 hours of irradiation, and in some cases after 12 hours. The general process used was that of Example 4, but with the photocatalyst indicated in Table 6. The photocatalyst was used at 0.5 mol % loading, and 150 mM of dichloromethane solvent. Table 6 also shows the results of each photocatalyst.

TABLE 6

| | | % | % | Time | E (kcal/mol) |
|---|---|---|---|---|---|
| | Photocatalyst | Cyclohexene | Urethane | (min) | (emissive) |
| Example | Ir(dFppy)₃ | 89 | 11 | 10 | 60.1 |
| 24 | | 4 | 93 | 240 | |
| Example | p-2CzPn | 93 | 6 | 10 | 60.3 |
| 25 | | 49 | 48 | 240 | |
| | | 43 | 53 | 720 | |
| Example | Ru(bpy)₃(PF₆)₂ | 100 | 0 | 240 | 46.5 |
| 26 | | | | | |
| Example | Ir(dtbu₃bpy)(dtbupppy)₂PF₆ | 100 | 0 | 10 | ? |
| 27 | | 94 | 6 | 240 | |
| Example | Ir(ppy)₃ | 97 | 3 | 10 | 55.2 |
| 28 | | 95 | 5 | 240 | |
| Example | Eosin Y | 100 | 0 | 240 | 45.5 |
| 29 | | | | | |
| Example | Ur(Ftbuppy)₃ | 75 | 23 | 10 | 59.4 |
| 30 | | 3 | 94 | 240 | |
| Example | 3,7-diphenyl-N-(1- | 100 | 0 | 240 | ? |
| 31 | naphthyl)-10-phenoxazine | | | | |
| Example | 2CzPN | 96 | 4 | 10 | 60.6 |
| 32 | | 40 | 60 | 240 | |
| Example | Ir(dFCF₃ppy)₂(dtbubpy)PF₆ | 93 | 7 | 10 | 60.1 |
| 33 | | 18 | 82 | 240 | |
| Example | Ir(4′-CF₃ppy)₃ | 97 | 3 | 10 | 56.4 |
| 34 | | 79 | 21 | 240 | |

It was hypothesized that bulkier ligands may mitigate catalyst degradation in the presence of amines, though at the risk of reducing energy transfer efficiency, but photocatalysts of 58 kcal/mol emissive energy or greater appeared to be essentially interchangeable. Ir(dFppy)₃ (CAS No. 387859-70-3), 2CzPN (4,5-di(9H-carbazol-9-yl) phthalonitrile) and its isomer, Ir(Ftbppy)₃, and Ir(dFCF₃ppy)₂(dtbbpy)PF₆ each functioned well at comparable efficiency, with the 2CzPN isomers operating slightly slower than the others. Ir (4′-CF₃ppy)₃, perhaps at the lower limit of emissive energy: 56.4 kcal/mol, was considerably less effective while catalysts of less than 56 kcal/mol emissive energy: Ru(bpy)₃(PF₆)₂, Ir(ppy)₃, Eosin Y, 3,7-diphenyl-N-(1-naphthyl)-10-phenoxazine yielded no products.

### Example 35

With general conditions in hand, a scope of over 30 primary and secondary aliphatic amines were tested in FIG. 6. For this example, 200 mM 4-fluorophenylcyclohexene and 200 mM of the amine were irradiated with blue light (using blue LEDs) in the presence of 0.5 mol % of Ir(Fppy)₃ in 0.1 MPa CO₂ and 200 mM of dichloromethane (CH₂Cl₂) solvent at 15° C. and atmospheric pressure. The results are reported in FIG. 6, with the yields of urethane reported under each amine pictured.

A wide range afforded isolated yields exceeding 60% despite using conditions optimized for benzylamine, with some exceptions having been exposed to modified conditions. Gaseous amines are commercially available as aqueous and ethanolic (amine #6, #11) solutions. While excellent yields were obtained even when added as aqueous solution, as was the case for amine #11, the yield was significantly reduced when methylamine was added as an ethanolic solution. This issue was solved when methylamine was first precipitated as the carbamate salt and decanted prior to addition to the reaction mixture for amine #6. Some amines bearing hydroxyl groups, such as R-phenylglycinol (amine #10), were poorly soluble in dichloromethane when exposed to CO₂ and were instead reacted in acetonitrile-water solution. A moderate amount of the tertiary alcohol was observed. Three primary phenomena were encountered with other amines that account for decreased reaction yields and, in some cases, altogether disabled reactivity: photocatalyst degradation, insolubility of the carbamate salt, and sluggish reactivity that poorly competes with cycloaddition. Arylamines failed to form any urethane under these conditions.

### Example 36

Also impractical, but of importance, was the question of whether CO₂ from a person's breath could facilitate reactivity. Optimizations up to this point indicated partial tolerance of low CO₂ pressure, partial O₂ contamination, and moisture. Human exhalation averages 4% composition CO₂. A reaction was accordingly set up with N-methylbenzylamine using a balloon of a person's exhalation, refilled periodically to ensure constant levels of CO₂. The reaction was run with 0.5 mol % Ir(Fppy)₃, 200 mM 1,2-dichloroethane, 200 mM 4-fluorophenylcyclohexene and 200 mM of the amine under blue light. This reaction resulted in 20% conversion to urethane, with 57% cyclohexene, 10% of the dimer, and 8% of cyclohexanol after 360 minutes.

It was a qualified success, approaching 43% conversion before reaching a standstill. With the photocatalyst and cyclohexene still present in the reaction mixture, it appeared that the amine had degraded in the presence of oxygen overnight, which might have been improved with more intermittent exhalations. While tolerance to oxygen has been demonstrated for alkene isomerization, it suggests that the reactive alkene is indeed a ground state singlet that is highly strained, as a long-lived triplet would be expected to react with the oxygen that is present.

### Example 37

With the desire to probe the significance of the basicity of the trans-cyclohexenes toward the reaction rate, a series of different cyclohexenes were investigated in FIG. 7. Set up in seven parallel experiments, 4-(trifluoromethyl)benzylamine was treated to cyclohexenes bearing various para-substituents, Ir(Fppy)₃, and CO₂. The reactions were monitored by ¹⁹F NMR over the course of visible-light irradiation to measure the relative initial rates of reaction normalized to phenylcyclohexene. These ratios were then converted to logarithms and plotted as a function of Hammett parameter σ. Entries 1 and 5 had to be removed from the plot since they exhibited rates of 2+2 cycloaddition that far exceeded the rates of urethane formation, such that none was observed to form. It appears that electron-withdrawing substituents that extend the conjugation of the x-system around the aromatic ring accelerate the undesired dimerization reaction. The other entries indicated a clear negative slope with significant sensitivity to the functional group changes, consistent with alkene protonation, a buildup of positive charge through the conjugated system, being part of the rate-determining step. This is also consistent with the much-improved reaction rate compared to the work of Yoshida and Inoue using less basic vinyl ethers.

### Example 38

This example showed the conversion of the formed arylcyclohexyl urethanes to more general carbamates or other useful derivatives. Transcarbamoylations was used to convert the arylcyclohexyl to other urethanes.

Example 38 (a): Urethanes produced by the process of Example 3 were mixed at room temperature with 1.5 equivalent of a KOt-bu {potassium tert-butoxide-[(CH₃)₃COK]ₙ-base} and a suitable alcohol (cinnamyl alcohol) in toluene to produce the cinnamyl carbamate and an arylcyclohexanol. This resulted in about a 56% conversion to cinnamyl carbamate.

Next, the arylcyclohexanol formed as the byproduct underwent dehydration to regenerate the cycloalkene by gentle heating (60° C. to 70° C.) with p-toluenesulfonic acid.

Example 38 (b): Urethane (250 mg, 809 μmol)—produced by the process of Example 3—were stirred with β-citronellol (380 mg, 2.4 mmol) and potassium tert-butoxide (136 mg, 1.2 mmol) in 2 mL DMSO at room temperature for approximately 2 hours, before being quenched with saturated aqueous NH₄Cl. The quenched reaction mixture was extracted into ethyl acetate (“EtOAc”; 3×1.5 mL). The combined EtOAc layers were subsequently washed with brine (5×1 mL) to remove DMSO before drying over MgSO₄ and concentrating down to a crude residue (612 mg). The residue was subjected to silica gel column chromatography (slow gradient toward 10% EtOAc in hexane) obtaining 1-phenylcyclohexanol and, separately, the urethane:

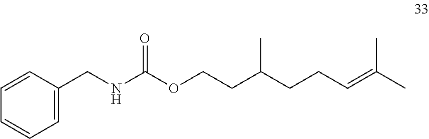 [SMILES: CC(C)=CCCC(C)CCOC(=O)NCc1ccccc1]

The urethane was a colorless oil (198 mg, 85% yield).

The cyclohexanol was subjected to acidic dehydrating conditions to obtain the corresponding cyclohexene (105 mg, 83% yield).

Example 38 (c): To obtain the urethane:

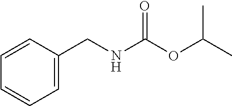

Urethanes (100 mg, 324 μmol)—from the process of Example 3—was stirred with 2-propanol (60 mg, 1 mmol) and potassium tert-butoxide (55 mg, 485 μmol) in 2 mL DMSO at room temperature for approximately 8 hours, before being quenched with saturated aqueous NH₄Cl. The quenched reaction mixture was extracted into EtOAc (3×1.5 mL). The combined EtOAc layers were subsequently washed with brine (5×1 mL) to remove DMSO before drying over MgSO₄ and filtering into a test tube. To the test tube was added about 20 mg (100 μmol) of p-toluenesulfonic acid monohydrate before placing in an oil bath for 30 minutes of reflux. The tube contents were then cooled to room temperature and quenched with 20 mg of triethylamine (200 μmol) before concentrating in vacuo to a brown residue. The residue was extracted into 3 mL of hexane and filtered through a silica plug with 1 additional mL of hexane to afford a cyclohexene (47 mg, 92% yield). The remaining tube residue was then extracted into EtOAc (3×1 mL) before being eluted through the silica gel plug into another receptacle with an additional 2 mL of clean EtOAc, isolating product 34 as a light brown low-melting solid after concentration under vacuum (58 mg, 93% yield).

As can be seen from the above examples, the present disclosure provides for mild and convenient conditions to create urethanes from a variety of primary and secondary aliphatic amines in good yields. Even though the resulting urethanes are still limited to arylcyclohexyl moieties as the O-substituents. They can be conveniently and readily converted to more general carbamates or other useful derivatives.

In summary, this disclosure illustrates a method for urethane synthesis by addition of CO₂ and amines to photoactivated olefins using arylcyclohexene under mild conditions, such as 0.1 MPa CO₂ at 7° C. (280 K). The method uses no phosgene, CO, or any reagents directly or indirectly derived from them. The method is generally water tolerant and boasts moderate to excellent yields for a broad range of aliphatic primary and secondary amines. While this method has limitations (i.e. arylamine and O-cyclohexyl requirement), it introduces the use of arylcyclohexenes as a strategy to exploit the potential energy of carried by photons to drive an endergonic process, CO₂-aminoalkylation. Furthermore, the process can be carried out in a step-wise fashion to accomplish CO₂-valorization while regenerating the arylcyclohexene via thermodynamically driven catalysis.

Aspects of this disclosure invention can be further understood from the following numbered embodiments.

Embodiment 1: A method of carbamate ester synthesis, the method comprising:
providing a first component which is an amine, a carbamate salt produced from the amine, or a mixture thereof;
providing an arylcycloalkene, and optionally an arylcyclohexene;
reacting the first component and arylcycloalkene by the addition of CO₂, a photocatalyst, and light to produce an arylcycloalkyl carbamate ester.

Embodiment 2: The method according to Embodiment 1, wherein the arylcycloalkene is arylcyclohexene, and the method produces arylcyclohexyl carbamate ester.

Embodiment 3: The method according to Embodiment 1 or Embodiment 2, further comprising:
providing an alcohol having a hydroxyl group and an alkane group;
treating the arylcycloalkyl carbamate ester with a base in the presence of the alcohol to produce an alkane carbamate ester and an arylcycloalkanol.

Embodiment 4: The method according to Embodiment 3, further comprising:
dehydrating the arylcycloalkanol in the presence of an acid to regenerate the arylcycloalkene.

Embodiment 5: The method according to any of Embodiments 1 to 4, wherein the photocatalyst is one that facilitates the reaction of the amine and arylcyclohexene by absorption of visible light, and more typically of light from 380 nm to 500 nm; and optionally from 455 nm to 380 nm.

Embodiment 6: The method according to Embodiment 5, wherein the photocatalyst is selected from the group consisting of (dFppy)₃, 2CzPN (4,5-di(9H-carbazol-9-yl) phthalonitrile), p-2CzPN, Ir(Ftbppy)₃, Ir(dFCF₃ppy)₂(dtbbpy)PF₆, and mixtures thereof.

Embodiment 7: The method according to any of Embodiments 1 to 6, wherein the step of reacting is carried out in a solvent selected from dichloromethane and acetonitrile.

Embodiment 8: The method according to any of Embodiments 1 to 7, wherein the step of reacting is carried out from −60° C. to 100° C. from 0.04 atm to 4 amt CO₂.

Embodiment 9: The method according to any of Embodiments 1 to 7, wherein the step of reacting is carried out at near standard temperature and pressure.

Embodiment 10: The method according to any of Embodiments 1 to 9, wherein the photocatalyst is present from 0.02 mol % to about 5 mol %, and optionally, from 0.05 mol % to 2 mol %, and optionally from 0.1 mol % to 1 mol %.

Embodiment 11: The method according to any of Embodiments 1 to 10, wherein the first component is an amine.

Embodiment 12. The method according to Embodiment 11, wherein the amine is a primary or secondary aliphatic amine.

Embodiment 13: The method according to Embodiment 12, wherein the amine and arylcycloalkene are present in a ration of from 1 to 2 or from 2 to 1, and optionally from 1 to 1.05 mole of amine per mole of arylcycloalkene.

Therefore, the present compositions and methods are well adapted to attain the ends and advantages mentioned, as well as those inherent therein. The particular examples disclosed above are illustrative only, as the present methods may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative examples disclosed above may be altered or modified, and all such variations are considered within the scope and spirit of the present treatment additives and methods. While compositions and methods are described in terms of “comprising,” “containing,” “having,” or “including” various components or steps, the compositions and methods can also, in some examples, “consist essentially of” or “consist of” the various components and steps. Whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee.

1. A method of carbamate ester synthesis, the method comprising:
providing a first component which is an amine, a carbamate salt produced from the amine, or a mixture thereof;
providing an arylcyclohexene;
reacting the first component and arylcyclohexene by the addition of CO₂, a photocatalyst, and light to produce an arylcyclohexyl carbamate ester.

2. The method according to claim 1, further comprising:
providing an alcohol having a hydroxyl group and an alkane group;
treating the arylcyclohexyl carbamate ester with a base in the presence of the alcohol to produce an alkane carbamate ester and an arylcyclohexanol.

3. The method according to claim 2, further comprising:
dehydrating the arylcyclohexanol in the presence of an acid to regenerate the arylcyclohexene.

4. The method according to claim 1, wherein the photocatalyst is one that facilitates the reaction of the amine and arylcyclohexene by absorption of visible light from 380 nm to 500 nm.

5. The method according to claim 4, wherein the photocatalyst is selected from the group consisting of (dFppy)₃, 2CzPN (4,5-di(9H-carbazol-9-yl) phthalonitrile), p-2CzPN, Ir(Ftbppy)₃, Ir(dFCF₃ppy)₂ (dtbbpy)PF₆, and mixtures thereof.

6. The method according to claim 5, wherein the photocatalyst is present from 0.02 mol % to about 5 mol %.

7. The method according to claim 4, wherein the first component is an amine.

8. The method according to claim 7, wherein the amine is a primary or secondary aliphatic amine.

9. The method according to claim 8, wherein the amine and arylcyclohexene are present in a ratio of from 1 to 2 or from 2 to 1.

10. The method according to claim 8, wherein the step of reacting is carried out from −60° C. to 100° C. from 0.04 atm to 4 amt CO₂.

11. The method according to claim 8, wherein the step of reacting is carried out at near standard temperature and pressure.

12. The method according to claim 11, wherein the step of reacting is carried out in a solvent selected from dichloromethane and acetonitrile.

13. A method of carbamate ester synthesis, the method comprising:
providing a first component which is a primary or secondary aliphatic amine;
providing an arylcyclohexene;
reacting the first component and arylcyclohexene by the addition of CO₂, a photocatalyst, and light to produce an arylcyclohexyl carbamate ester, and wherein the photocatalyst is one that facilitates the reaction of the amine and arylcyclohexene by absorption of visible light from 380 nm to 500 nm.

14. The method according to claim 13, further comprising:
providing an alcohol having a hydroxyl group and an alkane group;
treating the arylcyclohexyl carbamate ester with a base in the presence of the alcohol to produce an alkane carbamate ester and an arylcyclohexanol.

15. The method according to claim 14, further comprising:
dehydrating the arylcyclohexanol in the presence of an acid to regenerate the arylcyclohexene.

16. The method according to claim 13, wherein the photocatalyst is one that facilitates the reaction of the amine and arylcyclohexene by absorption of visible light from 455 nm to 380 nm.

17. The method according to claim 16, wherein the photocatalyst is selected from the group consisting of (dFppy)₃, 2CzPN (4,5-di(9H-carbazol-9-yl)phthalonitrile), p-2CzPN, Ir(Ftbppy)₃, Ir(dFCF₃ppy)₂ (dtbbpy)PF₆, and mixtures thereof.

18. The method according to claim 17, wherein the step of reacting is carried out at near standard temperature and pressure.

19. The method according to claim 18, wherein the photocatalyst is present from 0.05 mol % to 2 mol %.

20. The method according to claim 19, wherein the amine and arylcyclohexene are present in a ratio of from 1 to 1.05 mole of amine per mole of arylcyclohexene.
